(19) United States
(12) Patent Application Publication
(10) Pub. No.: US 2026/0242333 A1
(43) Pub. Date: Aug. 20, 2026

(54) METHOD AND DEVICE FOR PRODUCING ISOCYANATES

(71) Applicant: Covestro Deutschland AG, Leverkusen (DE)

(72) Inventors: Ruediger Borrmann, Köln (DE); Dieter Schade, Bergisch Gladbach (DE); Frank Richter, Leverkusen (DE); Tim Loddenkemper, Dormagen (DE)

(21) Appl. No.: 18/876,691

(22) PCT Filed: Jun. 20, 2023
(86) PCT No.: PCT/EP2023/066615

§ 371 Date: Dec. 19, 2024

(30) Foreign Application Priority Data
Jun. 22, 2022 (EP) 22180310.9

(51) Int. Cl. C07C 263/10

(52) U.S. Cl. CPC C07C 263/10; C07C 2601/14

(57) ABSTRACT

The invention relates to a method for producing isocyanates via gas-phase phosgenation of the corresponding amines in a phosgenation system, in which, in particular, problems due to the formation of deposits in apparatus of the phosgenation system, e.g. in particular the reaction section, during the initial operation (start-up) of the method are preferably avoided. The invention also relates to a device for carrying out the method according to the invention, and to a method for starting up a phosgenation system.

The invention relates to a process for producing isocyanates by gas phase phosgenation of the corresponding amines in a phosgenation plant, in which, in particular, problems owing to the formation of deposits in apparatuses in the phosgenation plant, such as the reaction zone in particular, are very substantially avoided during the startup of the process. The invention further relates to an apparatus for performance of the process of the invention and to a process for startup of a phosgenation plant.

Industrial scale production of isocyanates by reacting the corresponding amines with phosgene has long been known from the prior art, where the reaction can be conducted continuously or batchwise in the gas phase or liquid phase. Of particular significance are di-or polyisocyanates that are suitable for production of polymers, for example polyurethanes, polythiourethanes, polyisocyanurates or polyureas. The isocyanates used here are either aromatic isocyanates, for example methylene diphenyl diisocyanate or higher homologs thereof or tolylene diisocyanate, or aliphatic isocyanates, for example hexamethylene diisocyanate, pentamethylene diisocyanate or isophorone diisocyanate.

Modern industrial scale production of polyisocyanates is continuous. This is subject to recurrent planned shutdowns or else unplanned outages in the process. The reasons for this are various, and range from failure of apparatuses or control devices through disruptions in raw material supply to economic reasons, such as weak demand for the product.

WO2015/144658 A1 is concerned explicitly with such variances from normal operation, and gives pointers as to how, in the event of such variances, the risk of deposits in the mixing zone for mixing of amine and phosgene or in the reaction zone disposed downstream thereof for flow purposes can be minimized. For this purpose, it is recommended that an excess of phosgene over the amine always be ensured, for example by first heating up the reactor only with solvent and phosgene in the startup of continuous production and only then commencing the amine feed, or, in the case of shutdown of continuous production, first stopping the amine feed and only then the phosgene feed.

In the apparatuses upstream of the mixing zone and the reaction zone, which serve for preparation of the amine, i.e., for example, for the adjustment of temperature, evaporation or superheating thereof, however, there are problems in the event of a plant shutdown that are not discussed in the documents cited. For example, there is a number of amines that solidify at room temperature, such that, firstly, restarting after a shutdown is made considerably more difficult and, secondly, the cleaning of apparatuses containing such amines, for example for maintenance purposes, is made much more difficult.

WO2015/144681 A1 is concerned specifically with the startup of a gas phase phosgenation plant. It is disclosed here that the startup of such a plant is a critical process step. It is stated that there can be formation of amine droplets and of solids such as polyureas or amine hydrochlorides, resulting in an increase in the pressure differential required for sufficient flow through the plant. For the minimization of these problems, it is recommended in particular, even during the startup operation, that the reactant streams be matched to one another such that there is always a stoichiometric excess of phosgene in relation to the primary amino groups of the amine.

Furthermore, in operational practice, specifically for the phosgenation of amines in the gas phase, it has been found that there are amines that more commonly present problems under less optimal phosgenation conditions as exist in the startup of the process, i.e., for example, have a tendency to rapidly form deposits, and those with less problematic behavior under these process conditions, such as 1-amino-3,5,5-trimethyl-5-aminomethylcyclohexane (IPDA) in particular. Even in the case of consistent application of the teachings from WO2015/144681 A1 and WO2015/144658 A1, it is not possible to prevent a time delay of sometimes one hour or more before temperature profiles that exist in continuous operation have become established in the reaction zone. This is because (without being tied to any theory) the phosgenation reactor is heated up beforehand, but there is further heating owing to the exothermic reaction. In the initial phase, parts of the reaction zone are consequently not yet at optimal operating temperature, which, in phosgenation of comparatively problematic amines, can lead to early and irreversible formation of deposits. These deposits in turn can lead to a distinct rise in pressure, for example in the amine inlet of the amine apparatus. This cannot be resolved even by greater preheating of the reactor. Firstly, this is technically difficult to implement, and, secondly, the onset of exothermic reaction then leads to an excessive temperature in the reaction mixture and hence to damage to the product.

The phosgenation of mixtures of different amines where at least one amine contains an aliphatically bonded primary amino group is disclosed in DE2249459 A1. This involves phosgenation of mixtures of amines and then separation of one of the isocyanates from the reaction mixture. The economic benefit that arises from the better use of plant capacity in such a procedure specifically for particular isocyanates is pointed out. In all the reactions disclosed by way of example, a mixture of two or more amines is first produced in a fixed ratio and this mixture is then introduced into the phosgenation apparatus. For a continuous process, it is preferable to add the amines to the phosgene as a mixture or at least simultaneously. If the amines are initially charged as a mixture, the mixing ratio is firstly fixed, and the problems associated with more demanding amines secondly cannot be reliably avoided.

There is therefore a need for a process and apparatus for production of isocyanates that avoid the problems mentioned and permit an improved phosgenation process, especially during startup of the phosgenation plant, enable phosgenation of amines in variable mixing ratios and simplify the cleaning of apparatuses upstream of the phosgenation reactor by the amine route.

It was accordingly an object of the present invention to provide a process for producing isocyanates by gas phase phosgenation of the corresponding amines and a phosgenation plant for performance of that process, which in particular eliminate or at least reduce the aforementioned problems. In particular, the process and the apparatus were to avoid or at least distinctly reduce a rise in pressure (which usually occurs in the production of isocyanates from the corresponding amines that lead to such a rise in pressure in prior art plants and processes) in the apparatuses of the phosgenation plant, such as the amine stream inlet of the amine stream apparatus, in particular in the startup of the phosgenation plant. Such a rise in pressure was preferably to be avoided or delayed for as long as possible even during the production phase.

Taking account of this requirement, the present invention provides a process for producing isocyanates by gas phase phosgenation of the corresponding amines in a phosgenation plant comprising or consisting of
(a) at least one phosgene stream apparatus for provision of a gaseous phosgene stream optionally comprising, in addition to phosgene, an inert substance in the phosgene stream;
(b) optionally one inert substance stream apparatus for provision of an inert substance stream,
(c) at least one amine stream apparatus for provision of a gaseous amine stream optionally comprising, in addition to amines, an inert substance in the amine stream,
(d) a phosgenation reactor for mixing of the streams from (a) to (c) and reaction of the amine stream with the phosgene stream;
   wherein the phosgenation plant is started up by running through the following steps:
(A) charging the phosgenation reactor with the gaseous phosgene stream and optionally the inert substance stream, and then continuously feeding the gaseous phosgene stream and any inert substance stream into the phosgenation reactor,
(B) continuously feeding the amine stream into the phosgenation reactor,
   characterized in that step (B) comprises a startup phase, a subsequent reduction phase and a production phase that follows the reduction phase, wherein, during the startup phase, the amine stream comprises at least one first amine and optionally a second amine other than the first amine, and the proportion by mass of the first amine in the totality of amines in the amine stream which is fed to the phosgenation reactor is at least 90.0% by weight, then this proportion by mass is reduced during the reduction phase to 85.0% by weight or less, and then this proportion by mass is maintained in the production phase, wherein, during the startup phase, the reduction phase and the production phase, the remainder of the totality of the amines in the amine stream absent from 100% by weight is formed by the second amine and optionally to an extent of not more than 1% by weight of further organic amines other than the first and second amines, and wherein
the first amine is selected from the group comprising or consisting of 1,6-diaminohexane (HDA), 1-amino-3,5,5-trimethyl-5-aminomethylcyclohexane (IPDA), bis(p-aminocyclohexyl)methane (PACM) or mixtures thereof; and
the second amine is selected from the group comprising or consisting of 1,5-diaminopentane (PDA), 1,3-bis(aminomethyl)benzene (m-XDA), 1,4-bis(aminomethyl)benzene (p-XDA), 1,2-bis(aminomethyl)cyclohexane (1,2-BAC), 1,3-bis(aminomethyl)cyclohexane (1,3-BAC), 1,4-bis(aminomethyl)-cyclohexane (1,4-BAC), tetramethylxylylenediamine (TMXDA), 2,5-bis(aminomethyl)bicyclo[2.2.1]heptane (2,5-NBDA), 2,6-bis(aminomethyl)bicyclo[2.2.1]heptane (2,6-NBDA), neopentanediamine, 2,4,4-trimethylhexamethylenediamine, 2,2,4-trimethylhexamethylenediamine, 1,3-diaminocyclohexane, 1,4-diaminocyclohexane, 1-methyl-2,6-diaminocyclohexane and/or 1-methyl-2,4-diaminocyclohexane (collectively or individually H6TDA), 2,5-diamino-2,5-dideoxy-1,4:3,6-dianhydrosorbitol (ISODA), 2,5-diamino-2,5-dideoxy-1,4:3,6-dianhydromannitol (MANDA) and 2,5-diamino-2,5-dideoxy-1,4:3,6-dianhydroiditol (IDDA) or mixtures thereof.

The invention further relates to a phosgenation plant for performance of the process of the invention, comprising or consisting of:
(a) at least one phosgene stream apparatus for provision of a gaseous phosgene stream optionally comprising, in addition to phosgene, an inert substance in the phosgene stream;
(b) optionally one inert substance stream apparatus for provision of an inert substance stream;
(c) at least one amine stream apparatus for provision of a gaseous amine stream optionally comprising, in addition to amines, an inert substance in the amine stream; and
(d) a phosgenation reactor for mixing of the streams from (a) to (c) and reaction of the amine stream with the phosgene stream;
   characterized in that the amine stream apparatus is configured to provide the amine stream in such a way that, in a startup phase, the amine stream comprises at least one first amine and optionally a second amine, and the proportion by mass of the first amine in the totality of the amines in the amine stream that can be fed to the phosgenation reactor is at least 90.0% by weight, preferably 95.0% to 100% by weight, further preferably 97.5% to 100% by weight, more preferably 99.0% to 100.0% by weight, most preferably 100% by weight, then this proportion by mass may be reduced during a reduction phase that follows the startup phase to 85.0% by weight or less, preferably 0% to 80.0% by weight, further preferably 0.1% to 50.0% by weight, more preferably to 1.0% to 40.0% by weight, most preferably to 5.0% to 30.0% by weight, and then this proportion by mass may be maintained in a production phase that follows the reduction phase, wherein, during the startup phase, the reduction phase and the production phase, the remainder of the totality of the amines in the amine stream absent from 100% by weight is formed by the second amine and optionally to an extent of not more than 1% by weight, preferably not more than 0.2% by weight, of further organic amines other than the first and second amines.

The invention is also directed to a process for startup of a phosgenation plant comprising or consisting of
(a) at least one phosgene stream apparatus for provision of a gaseous phosgene stream optionally comprising, in addition to phosgene, an inert substance in the phosgene stream;
(b) optionally one inert substance stream apparatus for provision of an inert substance stream;
(c) at least one amine stream apparatus for provision of a gaseous amine stream optionally comprising, in addition to amines, an inert substance in the amine stream; and
(d) a phosgenation reactor for mixing of the streams from (a) to (c) and reaction of the amine stream with the phosgene stream;
   wherein the phosgenation plant is started up by running through the following steps:
(A) charging the phosgenation reactor with the gaseous phosgene stream and optionally the inert substance stream, and then continuously feeding the gaseous phosgene stream and any inert substance stream into the phosgenation reactor,
(B) continuously feeding the amine stream into the phosgenation reactor,
   characterized in that step (B) comprises a startup phase, a subsequent reduction phase and a production phase that follows the reduction phase, wherein, during the startup phase, the amine stream comprises at least one first amine and optionally a second amine other than the first amine, and the proportion by mass of the first amine in the totality of amines in the amine stream which is fed to the phosgenation reactor is at least 90.0% by weight, then this proportion by mass is reduced during the reduction phase to 85.0% by weight or less, and then this proportion by mass is maintained in the production phase, wherein, during the startup phase, the reduction phase and the production phase, the remainder of the totality of the amines in the amine stream absent from 100% by weight is formed by the second amine and optionally to an extent of not more than 1% by weight of further organic amines other than the first and second amines, and
the first amine is selected from the group comprising or consisting of 1,6-diaminohexane (HDA), 1-amino-3,5,5-trimethyl-5-aminomethylcyclohexane (IPDA), bis(p-aminocyclohexyl)methane (PACM) or mixtures thereof; and
the second amine is selected from the group comprising or consisting of 1,5-diaminopentane (PDA), 1,3-bis(aminomethyl)benzene (m-XDA), 1,4-bis(aminomethyl)benzene (p-XDA), 1,2-bis(aminomethyl)cyclohexane (1,2-BAC), 1,3-bis(aminomethyl)cyclohexane (1,3-BAC), 1,4-bis(aminomethyl)-cyclohexane (1,4-BAC), tetramethylxylylenediamine (TMXDA), 2,5-bis(aminomethyl)bicyclo[2.2.1]heptane (2,5-NBDA), 2,6-bis(aminomethyl)bicyclo[2.2.1]heptane (2,6-NBDA), neopentanediamine, 2,4,4-trimethylhexamethylenediamine, 2,2,4-trimethylhexamethylenediamine, 1,3-diaminocyclohexane, 1,4-diaminocyclohexane, 1-methyl-2,6-diaminocyclohexane and/or 1-methyl-2,4-diaminocyclohexane (collectively or individually H6TDA), 2,5-diamino-2,5-dideoxy-1,4:3,6-dianhydrosorbitol (ISODA), 2,5-diamino-2,5-dideoxy-1,4:3,6-dianhydromannitol (MANDA) and 2,5-diamino-2,5-dideoxy-1,4:3,6-dianhydroiditol (IDDA) or mixtures thereof.

The word “a” in the context of this invention, in connection with countable parameters, should be understood merely as the indefinite article and only as the number “one” when this is stated explicitly, for instance by the addition “exactly one”. Expressions such as “a heat exchanger” or “an amine stream” thus do not rule out the possibility of existence of further heat exchangers or amine streams.

The term “gaseous” should be considered also to include entrainment of droplets.

### Phosgenation Plant:

The phosgenation plant comprises or consists of
(a) at least one phosgene stream apparatus for provision of a gaseous phosgene stream optionally comprising, in addition to phosgene, an inert substance in the phosgene stream;
(b) optionally one inert substance stream apparatus for provision of an inert substance stream;
(c) at least one amine stream apparatus for provision of a gaseous amine stream optionally comprising, in addition to amines, an inert substance in the amine stream; and
(d) a phosgenation reactor for mixing of the streams from (a) to (c) and reaction of the amine stream with the phosgene stream.

The phosgene stream apparatus, the optional inert substance stream apparatus, the amine stream apparatus and the phosgenation reactor may mutually independently comprise further apparatuses useful for the operation of the phosgenation plant, for example reservoir vessels for starting materials, and heat exchangers for controlling the temperature of or optionally evaporating starting materials, and devices for processing of the reaction mixture after completed reaction of amine with phosgene.

The phosgene stream apparatus preferably comprises at least one heat exchanger for heating of the phosgene stream and/or the inert substance stream.

The amine stream apparatus is configured to provide the amine stream in such a way that, in a startup phase, the amine stream comprises at least one first amine and optionally a second amine, and the proportion by mass of the first amine in the totality of the amines in the amine stream that can be fed to the phosgenation reactor is at least 90.0% by weight, preferably 95.0% to 100% by weight, further preferably 97.5% to 100% by weight, more preferably 99.0% to 100.0% by weight, most preferably 100% by weight, then this proportion by mass may be reduced during a reduction phase that follows the startup phase to 85.0% by weight or less, preferably 0% to 80.0% by weight, further preferably 0.1% to 50.0% by weight, more preferably to 1.0% to 40.0% by weight, most preferably to 5.0% to 30.0% by weight, and then this proportion by mass may be maintained in a production phase that follows the reduction phase, wherein, during the startup phase, the reduction phase and the production phase, the remainder of the totality of the amines in the amine stream absent from 100% by weight is formed by the second amine and optionally to an extent of not more than 1% by weight of further organic amines other than the first and second amines.

In a preferred embodiment, the amine stream apparatus comprises a metering device for mutually independently variable provision of the amines in terms of proportions by mass. In other words, the amine stream apparatus preferably comprises one metering device per amine (or amine mixture if, for example, the first or second amine is already itself an amine mixture), such that the mass flow rates/proportions by mass can be controlled mutually independently. These may be, for example, metering pumps or closed-loop control valves.

Furthermore, it is preferable that the amine stream apparatus comprises
i. at least one reservoir vessel each for the first amine and the second amine,
ii. at least one metering device downstream of each of the reservoir vessels,
iii. optionally at least one mixing device downstream of the metering devices,
iv. optionally at least one evaporator for evaporation of the amine stream,
v. optionally a heat exchanger for heating of the amine stream.

The optional mixing device may, for example, be a T-piece in the pipeline, a mixing chamber, a static mixer or a pump, especially mixing pump, or stirred vessel. However, the amine streams from the metering devices can also be mixed in the evaporator. An evaporator is used in the case of liquid amines in particular.

The optional further organic amines other than the first and second amines may either be provided via a separate reservoir vessel or mixed with the first and/or second amine. In particular, this also includes impurities that may be present in the first and/or second amine. The amine stream apparatus preferably comprises, in at least one of the amine-conducting pipelines, at least one flange connection prepared for accommodation of a blank. This enables reliable prevention of amine flow to the corresponding conduit by means of the blank or reliable severing of the corresponding amine conduit.

Preferably at least one reservoir vessel and more preferably each reservoir vessel is set up to be heatable. This results in the advantage that the efficiency of the plant and of the process are further improved.

The phosgene stream apparatus, the optional inert substance stream apparatus and the amine stream apparatus are each connected to the phosgenation reactor via at least one inlet. The connection of the inlets to the phosgenation reactor may, for example, be via simple pipelines, flange connections, smooth jet nozzles, annular gap nozzles or a mixing element. The annular gap nozzle may be equipped here with two or more channels, preferably two in a coaxial arrangement, for the amine stream and the phosgene stream or an inert gas stream, where the amine stream and the phosgene stream do not meet until within the phosgenation reactor. Alternatively, especially in industrial scale application, the amine is added to the phosgenation reactor via an inlet tube, at the end of which there is a central nozzle and which is kept in position by a cover and optional holder/flow straightener, such that an annular space is formed around the inlet tube in the reactor, while the phosgene enters the ring space via a separate inlet and thence reaches the phosgenation reactor via the flow straightener (see also EP1362847 A2). The phosgenation reactor has a mixing zone where the streams meet for the first time, and a reaction zone where the streams are converted further. The mixing zone and reaction zone are preferably in one apparatus. In addition, it is preferable that the phosgenation reactor is essentially upright, and this is preferably connected at the upper end to the phosgene stream apparatus and the amine stream apparatus.

The phosgenation plant also preferably comprises at least one quench for cooling and at least partial condensation of the reaction mixture, which is connected to the phosgenation reactor, preferably at the lower end, where the quench is designed in particular for introduction of chlorobenzene, o-dichlorobenzene and/or at least one of the isocyanates produced in the phosgenation plant. In particular, the quench has a dedicated collecting vessel, where the collecting vessel is preferably incorporated in or separately connected to the quench.

### Startup of the Phosgenation Plant:

In the context of the present invention, the expression “startup of the phosgenation apparatus” encompasses all process steps required to convert a nonoperational phosgenation apparatus, for example after a maintenance-related shutdown, to continuous controlled operation for phosgenation of the corresponding amine(s).

In the context of the invention, the phosgenation plant is started up by running through at least the following steps:
(A) charging the phosgenation reactor with the gaseous phosgene stream and optionally the inert substance stream, and then continuously feeding the gaseous phosgene stream and any inert substance stream into the phosgenation reactor,
(B) continuously feeding the amine stream into the phosgenation reactor.

Step (B) comprises a startup phase, a subsequent reduction phase and a production phase that follows the reduction phase, wherein, during the startup phase, the amine stream comprises at least one first amine and optionally a second amine other than the first amine, and the proportion by mass of the first amine in the totality of amines in the amine stream which is fed to the phosgenation reactor is at least 90.0% by weight, then this proportion by mass is reduced during the reduction phase to 85.0% by weight or less, and then this proportion by mass is maintained in the production phase, wherein, during the startup phase, the reduction phase and the production phase, the remainder of the totality of the amines in the amine stream absent from 100% by weight is formed by the second amine and optionally to an extent of not more than 1% by weight of further organic amines other than the first and second amines.

In a preferred embodiment, step (A) is preceded by a preheating phase during which a heated inert gas, preferably nitrogen, is guided into the reactor through the amine stream inlet and/or the phosgene stream inlet.

In a preferred embodiment, the proportion by mass of the first amine in the totality of the amines in the amine stream during the startup phase is 95.0% to 100.0% by weight, preferably 97.5% to 100.0% by weight, more preferably 99.0% to 100.0% by weight, most preferably 100.0% by weight. In some cases, for example when the first amine and the second amine do not have a separate inlet to the reactor, this may result in disproportionate cleaning intensity to remove residues of other amines before the phosgenation apparatus is started up. Therefore, in such cases, the starting value for the proportion by mass of the first amine in the overall stream of amines fed to the phosgenation apparatus is not more than 99.9%, preferably not more than 99.7% and more preferably not more than 99.5%. The duration of the startup phase is preferably at least 5 minutes, preferably 10 to 2880 minutes, further preferably 15 to 1440 minutes, more preferably 20 to 240 minutes, most preferably 30 to 120 minutes.

During the reduction phase, it is preferable that the proportion by mass of the first amine in the totality of the amines in the amine stream is reduced to 0% to 80.0% by weight, further preferably to 0.1% to 50.0% by weight, more preferably to 1.0% to 40.0% by weight, most preferably to 5.0% to 30.0% by weight. It is further preferable that the proportion by mass of the first amine is reduced stepwise or continuously in the reduction phase. During the reduction phase, the proportion by mass of the first amine may even be temporarily reduced below the value which is maintained in the production phase. The duration of the reduction phase is preferably at least 0.01 minute, further preferably 0.1 to 240 minutes, more preferably 0.5 to 60 minutes, most preferably 1 to 30 minutes.

The phosgene stream and any inert substance stream, in the course of charging and continuous feeding in step (A), preferably have a temperature of 200° C. to 600° C., further preferably of 250° C. to 500° C., more preferably of 250° C. to 330° C. Step (A) may also be referred to as heating phase. In a preferred embodiment, the phosgene in the phosgenation reactor is present in a molar excess in relation to the amino groups of the amines of 30% to 330%, preferably 60% to 300% and more preferably 90% to 270%.

The amine stream, in the course of feeding into the phosgenation reactor in step (B), preferably has a temperature of 200° C. to 600° C., further preferably of 250° C. to 500° C., most preferably of 250° C. to 330° C. In a preferred embodiment, the feed of the amine stream is commenced only when the phosgenation reactor has been charged with phosgene and optionally inert substance and the desired starting temperature of 230° C. to 320° C., preferably 240° C. to 310° C., has been attained.

The inert substance is preferably selected from the group comprising or consisting of inert gases, inert solvents or mixtures of these. The inert gas may be selected from the group comprising or consisting of nitrogen, argon or mixtures of these. The solvent is preferably selected from the group comprising or consisting of aromatic hydrocarbons, preferably chlorobenzene, o-dichlorobenzene or mixtures of these.

If more than one amine is being reacted, it is possible first to mix the amines and then to evaporate and heat the amine mixture. Alternatively, one or more amines may be separately evaporated and heated. Different amines can also be fed separately into the phosgenation reactor through different feed devices (feeds), or the mixed amine stream can be fed in. Even in the case of separate feeding of two or more amines into the phosgenation reactor, the sum total of these amine streams is considered in the present context as the overall stream of amines being fed to the phosgenation reactor, such that proportions of the first amine in the overall stream of amines are based in each case on that overall stream of amines which is being fed to the reactor at a particular time, no matter whether as a mixture or in the form of a separate feed/inlet. The amines from separate reservoir vessels are preferably first mixed and then coevaporated, heated and fed into the phosgenation reactor. More preferably, the reservoir vessels are heated.

### Amines:

The first amine is selected from the group comprising or consisting of 1,6-diaminohexane (HDA), 1-amino-3,5,5-trimethyl-5-aminomethylcyclohexane (IPDA), bis(p-aminocyclohexyl)methane (PACM) or mixtures thereof.

The second amine is selected from the group comprising or consisting of 1,5-diaminopentane (PDA), 1,3-bis(aminomethyl)benzene (m-XDA), 1,4-bis(aminomethyl)benzene (p-XDA), 1,2-bis(aminomethyl)cyclohexane (1,2-BAC), 1,3-bis(aminomethyl)cyclohexane (1,3-BAC), 1,4-bis(aminomethyl)cyclohexane (1,4-BAC), tetramethylxylylenediamine (TMXDA), 2,5-bis(aminomethyl)bicyclo[2.2.1]heptane (2,5-NBDA), 2,6-bis(aminomethyl)bicyclo[2.2.1]heptane (2,6-NBDA), neopentanediamine, 2,4,4-trimethylhexamethylenediamine, 2,2,4-trimethylhexamethylenediamine, 1,3-diaminocyclohexane, 1,4-diaminocyclohexane, 1-methyl-2,6-diaminocyclohexane and/or 1-methyl-2,4-diaminocyclohexane (collectively or individually H6TDA), 2,5-diamino-2,5-dideoxy-1,4:3,6-dianhydrosorbitol (ISODA), 2,5-diamino-2,5-dideoxy-1,4:3,6-dianhydromannitol (MANDA) and 2,5-diamino-2,5-dideoxy-1,4:3,6-dianhydroiditol (IDDA) or mixtures thereof.

The optional further organic amines other than the first and second amines in the context of the inventions are primary, secondary and tertiary aliphatic, aromatic and heterocyclic amines. For example, further organic amines are 1-aminohexane, 1,2-diaminocyclohexane, 1,4-diaminocyclohexane, 6-aminocapronitrile, bis(hexamethylene)triamine, azepane, azepine, 2-aminomethylcyclopentylamine, 2-methylpentamethylenediamine, 2,3,4,5-tetrahydropyridine, piperidine, tri-n-butylamine or mixtures thereof.

### EMBODIMENTS

The present invention especially relates to the following embodiments:

In a first embodiment, the invention provides a process for producing isocyanates by gas phase phosgenation of the corresponding amines in a phosgenation plant comprising or consisting of
(a) at least one phosgene stream apparatus 20 for provision of a gaseous phosgene stream optionally comprising, in addition to phosgene, an inert substance in the phosgene stream;
(b) optionally one inert substance stream apparatus for provision of an inert substance stream,
(c) at least one amine stream apparatus 10 for provision of a gaseous amine stream optionally comprising, in addition to amines, an inert substance in the amine stream,
(d) a phosgenation reactor 1 for mixing of the streams from (a) to (c) and reaction of the amine stream with the phosgene stream;
   wherein the phosgenation plant is started up by running through the following steps:
(A) charging the phosgenation reactor 1 with the gaseous phosgene stream and optionally the inert substance stream, and then continuously feeding the gaseous phosgene stream and any inert substance stream into the phosgenation reactor 1,
(B) continuously feeding the amine stream into the phosgenation reactor 1,
   characterized in that step (B) comprises a startup phase, a subsequent reduction phase and a production phase that follows the reduction phase, wherein, during the startup phase, the amine stream comprises at least one first amine and optionally a second amine other than the first amine, and the proportion by mass of the first amine in the totality of amines in the amine stream which is fed to the phosgenation reactor 1 is at least 90.0% by weight, then this proportion by mass is reduced during the reduction phase to 85.0% by weight or less, and then this proportion by mass is maintained in the production phase, wherein, during the startup phase, the reduction phase and the production phase, the remainder of the totality of the amines in the amine stream absent from 100% by weight is formed by the second amine and optionally to an extent of not more than 1% by weight of further organic amines other than the first and second amines, and wherein
the first amine is selected from the group comprising or consisting of 1,6-diaminohexane (HDA), 1-amino-3,5,5-trimethyl-5-aminomethylcyclohexane (IPDA), bis(p-aminocyclohexyl)methane (PACM) or mixtures thereof; and
the second amine is selected from the group comprising or consisting of 1,5-diaminopentane (PDA), 1,3-bis(aminomethyl)benzene (m-XDA), 1,4-bis(aminomethyl)benzene (p-XDA), 1,2-bis(aminomethyl)cyclohexane (1,2-BAC), 1,3-bis(aminomethyl)cyclohexane (1,3-BAC), 1,4-bis(aminomethyl)cyclohexane (1,4-BAC), tetramethylxylylenediamine (TMXDA), 2,5-bis(aminomethyl)bicyclo[2.2.1]heptane (2,5-NBDA), 2,6-bis(aminomethyl)bicyclo[2.2.1]heptane (2,6-NBDA), neopentanediamine, 2,4,4-trimethylhexamethylenediamine, 2,2,4-trimethylhexamethylenediamine, 1,3-diaminocyclohexane, 1,4-diaminocyclohexane, 1-methyl-2,6-diaminocyclohexane and/or 1-methyl-2,4-diaminocyclohexane (collectively or individually H6TDA), 2,5-diamino-2,5-dideoxy-1,4:3,6-dianhydrosorbitol (ISODA), 2,5-diamino-2,5-dideoxy-1,4:3,6-dianhydromannitol (MANDA) and 2,5-diamino-2,5-dideoxy-1,4:3,6-dianhydroiditol (IDDA) or mixtures thereof.

In a second embodiment, the invention relates to a process according to embodiment 1, characterized in that the duration of the startup phase is at least 5 minutes, preferably 10 to minutes, further preferably 15 to 1440 minutes, more preferably 20 to 240, most preferably 30 to 120 minutes.

In a third embodiment, the invention relates to a process according to embodiment 1 or 2, characterized in that the reduction in the proportion by mass of the first amine in the reduction phase is stepwise or continuous.

In a fourth embodiment, the invention relates to a process according to any of the preceding embodiments, characterized in that the duration of the reduction phase is at least 0.01 minute, preferably at least 0.1 to 240 minutes, further preferably 0.5 to 60 minutes, more preferably to 30 minutes.

In a fifth embodiment, the invention relates to a process according to any of the preceding embodiments, characterized in that, during the startup phase, the proportion by mass of the first amine in the totality of the amines in the amine stream is 95.0% to 100.0% by weight, preferably 97.5% to 100.0% by weight, more preferably 99.0% to 100.0% by weight, most preferably 100.0% by weight.

In a sixth embodiment, the invention relates to a process according to any of the preceding embodiments, characterized in that, during the reduction phase, the proportion by mass of the first amine in the totality of the amines in the amine stream is reduced to 0% to 80.0% by weight, preferably to 0.1% to 50.0% by weight, further preferably to 1.0% to 40.0% by weight, more preferably to 5.0% to 30.0% by weight.

In a seventh embodiment, the invention relates to a process according to any of the preceding embodiments, characterized in that the phosgene stream and any inert substance stream, on charging and continuous feeding in step (A), are at a temperature of 200° C. to 600° C., preferably of 250° C. to 500° C., most preferably of 250° C. to 330° C.

In an eighth embodiment, the invention relates to a process according to any of the preceding embodiments, characterized in that the amine stream, on feeding into the phosgenation reactor 1 in step (B), is at a temperature of 200° C. to 600° C., preferably of 250° C. to 500° C., most preferably of 250° C. to 330° C.

In a ninth embodiment, the invention relates to a process according to any of the preceding embodiments, characterized in that the phosgene in the phosgenation reactor 1 is present in a molar excess in relation to the amino groups of the amines of 30% to 330%, preferably 60% to 300% and more preferably 90% to 270%.

In a tenth embodiment, the invention relates to a process according to any of the preceding embodiments, characterized in that the inert substance is selected from the group comprising or consisting of inert gases, inert solvents or mixtures of these.

In an eleventh embodiment, the invention relates to a process according to embodiment 10, characterized in that the inert substance is selected from the group comprising or consisting of inert gases, inert solvents or mixtures of these.

In a twelfth embodiment, the invention relates to a process according to embodiment 10 or 11, characterized in that the solvent is selected from the group comprising or consisting of aromatic hydrocarbons, preferably chlorobenzene, o-dichlorobenzene or mixtures of these. In a thirteenth embodiment, the invention relates to a phosgenation plant for performing the process according to the embodiments 1 to 12, comprising or consisting of:
(a) at least one phosgene stream apparatus 20 for provision of a gaseous phosgene stream optionally comprising, in addition to phosgene, an inert substance in the phosgene stream;
(b) optionally one inert substance stream apparatus for provision of an inert substance stream;
(c) at least one amine stream apparatus 10 for provision of a gaseous amine stream optionally comprising, in addition to amines, an inert substance in the amine stream; and
(d) a phosgenation reactor 1 for mixing of the streams from (a) to (c) and reaction of the amine stream with the phosgene stream;
   characterized in that the amine stream apparatus 10 is configured to provide the amine stream in such a way that, in a startup phase, the amine stream comprises at least one first amine and optionally a second amine, and the proportion by mass of the first amine in the totality of the amines in the amine stream that can be fed to the phosgenation reactor 1 is at least 90.0% by weight, preferably 95.0% to 100% by weight, further preferably 97.5% to 100% by weight, more preferably 99.0% to 100.0% by weight, most preferably 100% by weight, then this proportion by mass may be reduced during a reduction phase that follows the startup phase to 85.0% by weight or less, preferably 0% to 80.0% by weight, further preferably 0.1% to 50.0% by weight, more preferably to 1.0% to 40.0% by weight, most preferably to 5.0% to 30.0% by weight, and then this proportion by mass may be maintained in a production phase that follows the reduction phase, wherein, during the startup phase, the reduction phase and the production phase, the remainder of the totality of the amines in the amine stream absent from 100% by weight is formed by the second amine and optionally to an extent of not more than 1% by weight of further organic amines other than the first and second amines.

In a fourteenth embodiment, the invention relates to an apparatus according to embodiment 13, characterized in that the phosgene stream apparatus 20 comprises at least one heat exchanger 21 for heating of the phosgene stream and/or the inert substance stream.

In a fifteenth embodiment, the invention relates to an apparatus according to embodiment 13 or 14, characterized in that the amine stream apparatus 10 comprises a metering device 13 for mutually independently variable provision of the amines in terms of the proportions by mass.

In a sixteenth embodiment, the invention relates to an apparatus according to any of embodiments 13 to 15, characterized in that the amine stream apparatus 10 comprises
i. at least one reservoir vessel 11, 12 each for the first amine and the second amine, where the at least one reservoir vessel in each case is preferably set up to be heatable,
ii. at least one metering device 13 downstream of each of the reservoir vessels 11, 12,
iii. optionally at least one mixing device 14 downstream of the metering devices 13,
iv. optionally at least one evaporator 15 for evaporation of the amine stream,
v. optionally a heat exchanger 16 for heating of the amine stream.

In a seventeenth embodiment, the invention relates to an apparatus according to any of embodiments 13 to 16, characterized in that the phosgenation reactor 1 is essentially upright, and this is preferably connected at the upper end to the phosgene stream apparatus 20 and the amine stream apparatus 10.

In an eighteenth embodiment, the invention relates to an apparatus according to any of embodiments 13 to 17, characterized in that the phosgenation plant comprises at least one quench 2 for cooling and at least partial condensation of the reaction mixture, which is connected to the phosgenation reactor 1, preferably at the lower end, where the quench 2 is designed in particular for introduction of chlorobenzene, o-dichlorobenzene and/or at least one of the isocyanates produced in the phosgenation plant.

In a nineteenth embodiment, the invention relates to an apparatus according to embodiment 18, characterized in that the quench 2 has a dedicated collecting vessel, where the collecting vessel is especially incorporated in and/or separately connected to the quench.

In a twentieth embodiment, the invention relates to a process for startup of a phosgenation plant comprising
(a) at least one phosgene stream apparatus 20 for provision of a gaseous phosgene stream optionally comprising, in addition to phosgene, an inert substance in the phosgene stream;
(b) optionally one inert substance stream apparatus for provision of an inert substance stream;
(c) at least one amine stream apparatus 10 for provision of a gaseous amine stream optionally comprising, in addition to amines, an inert substance in the amine stream; and
(d) a phosgenation reactor 1 for mixing of the streams from (a) to (c) and reaction of the amine stream with the phosgene stream;
   wherein the phosgenation plant is started up by running through the following steps:
(A) charging the phosgenation reactor 1 with the gaseous phosgene stream and optionally the inert substance stream, and then continuously feeding the gaseous phosgene stream and any inert substance stream into the phosgenation reactor 1,
(B) continuously feeding the amine stream into the phosgenation reactor 1, characterized in that step (B) comprises a startup phase, a subsequent reduction phase and a production phase that follows the reduction phase, wherein, during the startup phase, the amine stream comprises at least one first amine and optionally a second amine other than the first amine, and the proportion by mass of the first amine in the totality of amines in the amine stream which is fed to the phosgenation reactor 1 is at least 90.0% by weight, then this proportion by mass is reduced during the reduction phase to 85.0% by weight or less, and then this proportion by mass is maintained in the production phase, wherein, during the startup phase, the reduction phase and the production phase, the remainder of the totality of the amines in the amine stream absent from 100% by weight is formed by the second amine and optionally to an extent of not more than 1% by weight of further organic amines other than the first and second amines, and
the first amine is selected from the group comprising or consisting of 1,6-diaminohexane (HDA), 1-amino-3,5,5-trimethyl-5-aminomethylcyclohexane (IPDA), bis(p-aminocyclohexyl)methane (PACM) or mixtures thereof; and
the second amine is selected from the group comprising or consisting of 1,5-diaminopentane (PDA), 1,3-bis(aminomethyl)benzene (m-XDA), 1,4-bis(aminomethyl)benzene (p-XDA), 1,2-bis(aminomethyl)cyclohexane (1,2-BAC), 1,3-bis(aminomethyl)cyclohexane (1,3-BAC), 1,4-bis(aminomethyl)cyclohexane (1,4-BAC), tetramethylxylylenediamine (TMXDA), 2,5-bis(aminomethyl)bicyclo[2.2.1]heptane (2,5-NBDA), 2,6-bis(aminomethyl)bicyclo[2.2.1]heptane (2,6-NBDA), neopentanediamine, 2,4,4-trimethylhexamethylenediamine, 2,2,4-trimethylhexamethylenediamine, 1,3-diaminocyclohexane, 1,4-diaminocyclohexane, 1-methyl-2,6-diaminocyclohexane and/or 1-methyl-2,4-diaminocyclohexane (collectively or individually H6TDA), 2,5-diamino-2,5-dideoxy-1,4:3,6-dianhydrosorbitol (ISODA), 2,5-diamino-2,5-dideoxy-1,4:3,6-dianhydromannitol (MANDA) and 2,5-diamino-2,5-dideoxy-1,4:3,6-dianhydroiditol (IDDA) or mixtures thereof.

In a twenty-first embodiment, the invention relates to use of an apparatus according to any of embodiments 13 to 19 for performance of a process according to any of embodiments 1 to 12.

### EXAMPLES

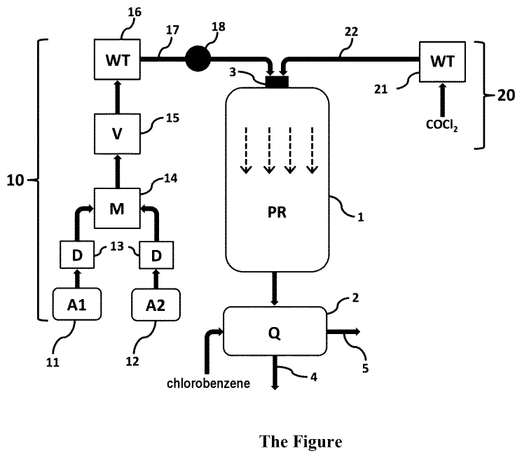

The present invention will now be elucidated with reference to FIG. 1 and the examples that follow, but is not limited thereto. The figure shows:

FIG. 1: a schematic diagram of a phosgenation plant of the invention.

FIG. 1 shows a schematic diagram of a phosgenation plant of the invention. This comprises an upright tubular reactor/phosgenation reactor (PR) 1 with a delay zone for reaction of an amine stream with a phosgene stream and a downstream quench (Q) with a collecting vessel 2 for cooling and at least partial condensation of the reaction gases formed in the phosgenation reactor 1, and an amine stream apparatus 10 for provision of the amine stream, and a phosgene stream apparatus 20 for provision of the phosgene stream. The quench 2 has a product outlet 4 for onward conduction of the liquid crude product, and a gas outlet 5 to lead off the offgases.

The amine stream apparatus 10 has a first and second reservoir vessel 11, 12 for a first amine (A1) and a second amine (A2) and a metering device (D) 13 downstream of each of the reservoir vessels 11, 12 for mutually independently variable adjustment of the ratios (or proportions by mass) of the amines (A1), (A2) from the first and second reservoir vessels 11, 12. Downstream of the metering device 13 is disposed a mixing device (M) 14 for mixing of the amine streams, where the mixing device 14 has a downstream evaporator (V) 15 for evaporation of the amine stream. Downstream of the evaporator 15 in turn is a heat exchanger (WT) 16 for heating of the amine stream.

The phosgene stream apparatus 20 comprises a heat exchanger (WT) 21 for heating of the phosgene stream.

The amine stream apparatus 10 is connected to the phosgenation reactor 1 via an amine stream inlet 17, and the phosgene stream apparatus 20 via a phosgene stream inlet 22, where the inlets open into an annular gap nozzle 3 which is provided in the top region of the phosgenation reactor 1 and has two channels in a coaxial arrangement. The separate feeding of the phosgene stream and the amine stream allows mixing and conversion of these reactants immediately after injection into the phosgenation reactor. The amine stream inlet 17 of the amine stream apparatus 10 is additionally equipped with a manometer 18 to measure the pressure in the amine stream inlet 17.

### General Procedure:

The examples that follow were conducted in a pilot plant for gas phase phosgenation according to FIG. 1. Phosgene was heated to about 340° C. in the heat exchanger 21 and introduced at that temperature into the phosgenation reactor 1 via the phosgene stream inlet 22 and the annular gap nozzle 3. On the amine side, as required, the first and/or second amine were each metered in mutually independently from the reservoir vessels 11 and 12 via a metering device 13, such that the desired proportions by mass are achieved. After passing through the metering device 13, the amines were mixed in a mixing device 14, then evaporated in an evaporator 15, then superheated to about 330° C. in a heat exchanger 16 and introduced at that temperature into the phosgenation reactor 1 via the amine stream inlet 17 and the annular gap nozzle 3. The molar phosgene excess for each amine or amine mixture used was between 140% and 300%, based on the amino groups of the amine. In order to start up the continuous reaction, the phosgenation reactor 1 was first charged solely with hot phosgene until a constant temperature had been established. Only then was the supply of the amine vapor commenced. The reaction was adiabatic apart from unavoidable heat losses. After passing through the delay zone, the reaction gases were cooled down and at least partly condensed in the quench 2 by introduction of chlorobenzene. The pressure in the amine stream inlet 17 was observed by means of a manometer 18.

### Comparative Example 1

The phosgenation reactor 1 was heated up to the starting temperature by means of phosgene vapor as described in the general procedure. Then metered addition of a 20:80 mixture (weight ratio) of ISODA to IPDA from the two amine reservoirs via the metering devices 13 into the mixing device 14 was commenced, and hence the phosgenation reaction was started. Deposits were formed in the phosgenation reactor 1, which led to a rapid and accelerating rise in pressure in the amine stream inlet 17 of the amine stream apparatus 10. After about 9 minutes, the pressure had risen from initially about 1000 mbar to 1500 mbar, and the reaction was ended.

### Example 1

The phosgenation reactor 1 was prepared as in comparative example 1. However, after the heating phase, in a startup phase, at first only IPDA was metered into the mixing device 14 via the metering devices 13, i.e. ISODA to IPDA=0:100 (weight ratio), in order to commence the reaction firstly with IPDA. After a continuous operating time of 60 minutes, the reduction phase was initiated. This involved reducing the delivery rate of IPDA and simultaneously commencing the metered addition of ISODA until a 10:90 mixture (weight ratio) of ISODA to IPDA was being reacted. After a further 30 minutes, the amine delivery rates were adjusted further, such that the weight ratio of ISODA to IPDA was then 20:80 (weight ratio), i.e. exactly the ratio from comparative example 1. Phosgenation was continued for a further 60 minutes at that mixing ratio (production phase), before the reaction was ended. The pressure in the amine stream inlet 17 of the amine stream apparatus 10 remained virtually constant during the reaction and was about 1020 mbar at the end (initially about 1000 mbar).

### Comparative Example 2

The phosgenation reactor 1 was heated up to the starting temperature by means of phosgene vapor as described in the general procedure. Then the reaction was started by evaporating PDA and running it into the phosgenation reactor 1 via the nozzle, corresponding to a weight ratio of PDA to IPDA of 100:0. A rapid rise in pressure in the amine stream inlet 17 of the amine stream apparatus 10 was observed, with attainment of the limit of 1500 mbar (initially about 1000 mbar) after only about 6 min, and so the reaction was ended.

### Example 2

The phosgenation reactor 1 was prepared as in comparative example 2. However, after the heating phase, at first only IPDA was metered into the mixing device 14 via the metering devices 13, i.e. PDA to IPDA=0:100 (weight ratio), in order to commence the reaction firstly with IPDA (startup phase). After 1.5 h, alteration of the weight ratio of PDA to IPDA in 5 equal steps (20:80, 40:60, 60:40, 80:20 and 100:0) was commenced every 30 minutes to the target value of 100:0 (weight ratio of PDA to IPDA) (reduction phase). The reaction was continued for a further 1.5 hours with PDA (production phase), before the experiment was ended at a pressure of 1090 mbar in the amine stream inlet 17 to the amine stream apparatus 10. The formation of deposits was thus considerably slowed compared to comparative example 2.

### LIST OF REFERENCE SYMBOLS

1 phosgenation reactor/tubular reactor (PR)
2 quench with collecting vessel (Q)
3 annular gap nozzle with two channels in coaxial arrangement
4 product outlet
5 gas outlet
10 amine stream apparatus
11 reservoir vessel for the first amine (A1)
12 reservoir vessel for the second amine (A2)
13 metering device(s)
14 mixing device (M)
15 evaporator (V) of the amine stream apparatus
16 heat exchanger (WT) of the amine stream apparatus
17 amine stream inlet
18 manometer
20 phosgene stream apparatus
21 heat exchanger (WT) of the phosgene stream apparatus
22 phosgene stream inlet

1. A process for producing isocyanates by gas phase phosgenation comprising:
(a) at least one phosgene stream apparatus (20) for provision of a gaseous phosgene stream optionally comprising, in addition to phosgene, an inert substance in the phosgene stream,
(b) optionally one inert substance stream apparatus for provision of an inert substance stream,
(c) at least one amine stream apparatus (10) for provision of a gaseous amine stream optionally comprising, in addition to amines, an inert substance in the amine stream,
(d) a phosgenation reactor (1) for mixing of the streams from (a) to (c) and reaction of the amine stream with the phosgene stream,
wherein the phosgenation plant is started up by running through the following steps:
(A) charging the phosgenation reactor (1) with the gaseous phosgene stream and optionally the inert substance stream, and then continuously feeding the gaseous phosgene stream and any inert substance stream into the phosgenation reactor (1),
(B) continuously feeding the amine stream into the phosgenation reactor (1),
wherein step (B) comprises a startup phase, a subsequent reduction phase and a production phase that follows the reduction phase, wherein, during the startup phase, the amine stream comprises at least one first amine and optionally a second amine other than the first amine, and the proportion by mass of the first amine in the totality of amines in the amine stream which is fed to the phosgenation reactor (1) is at least 90.0% by weight, then this proportion by mass is reduced during the reduction phase to 85.0% by weight or less, and then this proportion by mass is maintained in the production phase, wherein, during the startup phase, the reduction phase and the production phase, the remainder of the totality of the amines in the amine stream absent from 100% by weight is formed by the second amine and optionally to an extent of not more than 1% by weight of further organic amines other than the first and second amines, and wherein
the first amine is 1,6-diaminohexane (HDA), 1-amino-3,5,5-trimethyl-5-aminomethyl-cyclohexane (IPDA), bis(p-aminocyclohexyl)methane (PACM) or mixtures thereof; and
the second amine is 1,5-diaminopentane (PDA), 1,3-bis(aminomethyl)benzene (m-XDA), 1,4-bis(aminomethyl)benzene (p-XDA), 1,2-bis(aminomethyl)cyclohexane (1,2-BAC), 1,3-bis(aminomethyl)cyclohexane (1,3-BAC), 1,4-bis(aminomethyl)-cyclohexane (1,4-BAC), tetramethylxylylenediamine (TMXDA), 2,5 bis(aminomethyl)bicyclo[2.2.1]heptane (2,5-NBDA), 2,6 bis(aminomethyl)bicyclo[2.2.1]heptane (2,6-NBDA), neopentane-diamine, 2,4,4-trimethylhexamethylenediamine, 2,2,4-trimethyl-hexamethylenediamine, 1,3-diaminocyclohexane, 1,4-diaminocyclohexane, 1-methyl-2,6-diaminocyclohexane and/or 1-methyl-2,4-diaminocyclohexane (collectively or individually H6TDA), 2,5-diamino-2,5-dideoxy-1,4:3,6-dianhydrosorbitol (ISODA), 2,5-diamino-2,5-dideoxy-1,4:3,6-dianhydromannitol (MANDA) and 2,5-diamino-2,5-dideoxy-1,4:3,6-dianhydroiditol (IDDA) or mixtures thereof.

2. The process of claim 1, wherein the startup phase has a duration of 20 to 240 minutes, and the reduction phase has a duration of 0.5 to 60 minutes.

3. The process of claim 1, wherein during the startup phase, the proportion by mass of the first amine in the totality of the amines in the amine stream is 95.0% to 100.0% by weight.

4. The process of claim 1, wherein during the reduction phase, the proportion by mass of the first amine in the totality of the amines in the amine stream is reduced to 0.1% to 50.0% by weight.

5. The process of claim 1, wherein the phosgene stream and any inert substance stream, on charging and continuous feeding in step (A), are at a temperature of 200° C. to 600° C. or wherein the amine stream, on feeding into the phosgenation reactor (1) in step (B), is at a temperature of 200° C. to 600° C.

6. The process of claim 1, wherein the phosgene in the phosgenation reactor (1) is present in a molar excess in relation to the amino groups of the amines of 30% to 330%.

7. The process of claim 1, wherein the inert substance is inert gases, inert solvents or mixtures of these.

8. The process as claimed of claim 7, wherein the inert gas is nitrogen, argon or mixtures of these, and wherein the solvent is aromatic hydrocarbons, preferably chlorobenzene, o-dichlorobenzene or mixtures of these.

9. A phosgenation plant for performing the process of claim 1, comprising:
(a) at least one phosgene stream apparatus (20) for provision of a gaseous phosgene stream optionally comprising, in addition to phosgene, an inert substance in the phosgene stream;
(b) optionally one inert substance stream apparatus for provision of an inert substance stream;
(c) at least one amine stream apparatus (10) for provision of a gaseous amine stream optionally comprising, in addition to amines, an inert substance in the amine stream; and
(d) a phosgenation reactor (1) for mixing of the streams from (a) to (c) and reaction of the amine stream with the phosgene stream;
wherein the amine stream apparatus (10) is configured to provide the amine stream in such a way that, in a startup phase, the amine stream comprises at least one first amine and optionally a second amine, and the proportion by mass of the first amine in the totality of the amines in the amine stream that can be fed to the phosgenation reactor (1) is at least 90.0% by weight, then this proportion by mass may be reduced during a reduction phase that follows the startup phase to 85.0% by weight or less, and then this proportion by mass may be maintained in a production phase that follows the reduction phase, wherein, during the startup phase, the reduction phase and the production phase, the remainder of the totality of the amines in the amine stream absent from 100% by weight is formed by the second amine and optionally to an extent of not more than 1% by weight of further organic amines other than the first and second amines.

10. The apparatus of claim 9, wherein the phosgene stream apparatus comprises at least one heat exchanger (21) for heating of the phosgene stream and/or the inert substance stream.

11. The apparatus of claim 9, wherein the amine stream apparatus comprises a metering device (13) for mutually independently variable provision of the amines in terms of the proportions by mass.

12. The apparatus as claimed in claim 9, wherein the amine stream apparatus (10) comprises
i. at least one reservoir vessel (11, 12) each for the first amine and the second amine,
ii. at least one metering device (13) downstream of each of the reservoir vessels (11, 12),
iii. optionally at least one mixing device (14) downstream of the metering devices (13),
iv. optionally at least one evaporator (15) for evaporation of the amine stream,
v. optionally a heat exchanger (16) for heating of the amine stream.

13. The apparatus of claim 9, wherein the phosgenation plant comprises at least one quench (2) for cooling and at least partial condensation of the reaction mixture, which is connected to the phosgenation reactor (1), wherein the quench (2) is designed in particular for introduction of chlorobenzene, o-dichlorobenzene or at least one of the isocyanates produced in the phosgenation plant.

14. The apparatus of claim 13, wherein the quench (2) has a dedicated collecting vessel, where the collecting vessel is especially incorporated in or separately connected to the quench (2).

15. A process for startup of a phosgenation plant comprising:
(a) at least one phosgene stream apparatus (20) for provision of a gaseous phosgene stream optionally comprising, in addition to phosgene, an inert substance in the phosgene stream;
(b) optionally one inert substance stream apparatus for provision of an inert substance stream;
(c) at least one amine stream apparatus (10) for provision of a gaseous amine stream optionally comprising, in addition to amines, an inert substance in the amine stream; and
(d) a phosgenation reactor (1) for mixing of the streams from (a) to (c) and reaction of the amine stream with the phosgene stream;
wherein the phosgenation plant is started up by running through the following steps:
(A) charging the phosgenation reactor (1) with the gaseous phosgene stream and optionally the inert substance stream, and then continuously feeding the gaseous phosgene stream and any inert substance stream into the phosgenation reactor (1),
(B) continuously feeding the amine stream into the phosgenation reactor (1),
wherein step (B) comprises a startup phase, a subsequent reduction phase and a production phase that follows the reduction phase, wherein, during the startup phase, the amine stream comprises at least one first amine and optionally a second amine other than the first amine, and the proportion by mass of the first amine in the totality of amines in the amine stream which is fed to the phosgenation reactor (1) is at least 90.0% by weight, then this proportion by mass is reduced during the reduction phase to 85.0% by weight or less, and then this proportion by mass is maintained in the production phase, wherein, during the startup phase, the reduction phase and the production phase, the remainder of the totality of the amines in the amine stream absent from 100% by weight is formed by the second amine and optionally to an extent of not more than 1% by weight of further organic amines other than the first and second amines, and
the first amine is 1,6-diaminohexane (HDA), 1-amino-3,5,5-trimethyl-5-aminomethylcyclohexane (IPDA), bis(p-aminocyclohexyl)methane (PACM) or mixtures thereof; and
the second amine is 1,5-diaminopentane (PDA), 1,3-bis(aminomethyl)benzene (m-XDA), 1,4-bis(aminomethyl)benzene (p-XDA), 1,2-bis(aminomethyl)cyclohexane (1,2-BAC), 1,3-bis(aminomethyl)cyclohexane (1,3-BAC), 1,4-bis(aminomethyl)-cyclohexane (1,4-BAC), tetramethylxylylenediamine (TMXDA), 2,5bis(aminomethyl)bicyclo[2.2.1]heptane (2,5-NBDA), 2,6 bis(aminomethyl)bicyclo[2.2.1]heptane (2,6-NBDA), neopentanediamine, 2,4,4-trimethylhexamethylenediamine, 2,2,4-trimethyl-hexamethylenediamine, 1,3-diaminocyclohexane, 1,4-diaminocyclohexane, 1-methyl-2,6-diaminocyclohexane and/or 1-methyl-2,4-diaminocyclohexane (collectively or individually H6TDA), 2,5-diamino-2,5-dideoxy-1,4:3,6-dianhydrosorbitol (ISODA), 2,5-diamino-2,5-dideoxy-1,4:3,6-dianhydromannitol (MANDA) and 2,5-diamino-2,5-dideoxy-1,4:3,6-dianhydroiditol (IDDA) or mixtures thereof.

16. The process of claim 1, wherein during the reduction phase, the proportion by mass of the first amine in the totality of the amines in the amine stream is reduced to 1.0% to 40.0% by weight.

17. The process of claim 1, wherein during the reduction phase, the proportion by mass of the first amine in the totality of the amines in the amine stream is reduced to 5.0% to 30.0% by weight.

18. The process of claim 1, wherein the phosgene stream and any inert substance stream, on charging and continuous feeding in step (A), are at a temperature of 250° C. to 330° C., or wherein the amine stream, on feeding into the phosgenation reactor (1) in step (B), is at a temperature of 250° C. to 330° C.

19. The process of claim 1, wherein the phosgene in the phosgenation reactor (1) is present in a molar excess in relation to the amino groups of the amines of 60% to 300%.

20. The process of claim 1, wherein the phosgene in the phosgenation reactor (1) is present in a molar excess in relation to the amino groups of the amines of 90% to 270%.
